(19) United States
(12) Patent Application Publication
(10) Pub. No.: US 2021/0161470 A1
(43) Pub. Date: Jun. 3, 2021

(54) WEARABLE WELLNESS TRACKER

(71) Applicant: Pulsesol Digital Inc., Bellevue, WA (US)

(72) Inventors: Shiyuan WU, Shenzhen (CN); Peipei CHEN, Seattle, WA (US)

(21) Appl. No.: 17/012,853

(22) Filed: Sep. 4, 2020

(30) Foreign Application Priority Data
Nov. 29, 2019 (CN) 201922099282.6

(51) Int. Cl. A61B 5/00

(52) U.S. Cl. CPC A61B 5/6831; A61B 5/6825

(57) ABSTRACT

The utility model discloses a comfortable wearable human health index monitor, which includes a strap and a monitor host; a side of the band which is in contact with the human body is a textile, and the band has a receiving cavity therein. A window communicating with the accommodating cavity is opened on the side in contact with the human body; the monitor host is housed in the accommodating cavity, and the detection end of the monitor host is exposed through the window and is separated from the human body by textiles. By designing the side of the band that is in contact with the human body as a textile and cooperating with the monitor host in the receiving cavity, the textile can be used to separate the main body from the human body, effectively improving the breathability of the product and avoiding sweating, allergies, etc. that human skin is prone to, thus making the wearable more comfortable and very suitable for children.

### CROSS-REFERENCE TO RELATED APPLICATIONS

This application claims benefit of Chinese Application No. 201922099282.6, filed on Nov. 29, 2019, entitled “COMFORTABLE WEARABLE HUMAN HEALTH INDEX MONITOR,” the content of which is incorporated by reference herein in its entirety. This application also incorporates by reference for all purposes the full disclosure of Chinese Application No. 201930654177.7, filed on Nov. 26, 2019, entitled “BABY SLEEP RECORDER,” and Chinese Application No. 201930654163.5, filed on Nov. 26, 2019, entitled “BABY SLEEP RECORDER.”

### BACKGROUND

The present invention relates to the field of monitor technology and more particularly to a human health index monitor that is comfortable to wear.

With the rapid development of the economy and the continuous improvement of people's living standards, people pay more and more attention to daily health care and routine examinations, and health issues have increasingly become one of the key issues of social concern. At present, there are many types of equipment for physical health monitoring, which can monitor different physical indicators.

At present, wearable monitors have already appeared on the market. When used, they are worn on the human body (such as on the hands), which makes them very convenient.

However, these wearable monitors generally have the problem of poor breathability, which causes sweating, allergies, etc. which easily occur with human skin making wearables uncomfortable to wear and unsuitable for children. Therefore, improvements to current wearable monitors is necessary.

In view of this, the present invention addresses the lack of existing technology, and its main purpose is to provide a comfortable wearable human health index monitor, which can effectively solve the problem of discomfort when wearing existing wearable monitors.

In order to achieve the above purpose, the present invention adopts the following technical solution:

A comfortable wearable human health index monitor includes a band and a monitor host; wherein a side of the band that is in contact with the human body is a textile, the band having a receiving cavity therein, wherein the side that is in contact with the human body is provided with a window that communicates with the receiving cavity; wherein the monitor host is housed in the receiving cavity, and the detection end of the monitor host is exposed through the window and separated from the human body by textiles.

As a preferred solution, the band is entirely an elastic textile.

As a preferred solution, two ends of the band are fixedly connected by Velcro.

As a preferred solution, the receiving cavity is located at a middle position of the band.

As a preferred solution, the monitor host has a housing, wherein a bottom surface of the housing is a curved concave surface adapted to a human body, and wherein a detection end of the monitor host is located on the curved concave surface.

Compared with the prior art, the present utility model has obvious advantages and beneficial effects. Specifically, this can be known from the foregoing technical solutions:

By designing the side of the band that is in contact with the human body as a textile and cooperating with the monitor host in the receiving cavity, the textile can be used to separate the main body from the human body, effectively improving the breathability of the product and avoiding sweating, allergies, etc. that human skin is prone to, which makes it more comfortable to wear and very suitable for children.

In order to more clearly illustrate the structural features and effects of the present invention, the present invention will be described in detail below with reference to the drawings and specific embodiments.

### BRIEF DESCRIPTION OF THE DRAWINGS

FIG. 1 is a developed perspective view of a preferred embodiment of the present invention;

FIG. 2 is a schematic perspective development view of the preferred embodiment of the present invention from another angle;

FIG. 3 is a schematic perspective view of the preferred embodiment of the present invention when worn;

FIG. 4 is a schematic perspective view from another angle of the preferred embodiment of the present invention when worn;

FIG. 5 is a cross-sectional view of the preferred embodiment of the present invention when worn;

FIG. 6 is an enlarged schematic view of the monitor host in the preferred embodiment of the present invention; and

FIG. 7 is an enlarged schematic view from another angle of the monitor host in the preferred embodiment of the present invention.

### DRAWING IDENTIFICATION

| | | | |
|---|---|---|---|
| 10. | Band | 11. | Receiving cavity |
| 12. | Window | 13. | Velcro |
| 20. | Monitor host | 21. | Housing |
| 201. | Detection end | 202. | Detection end |

### DETAILED DESCRIPTION

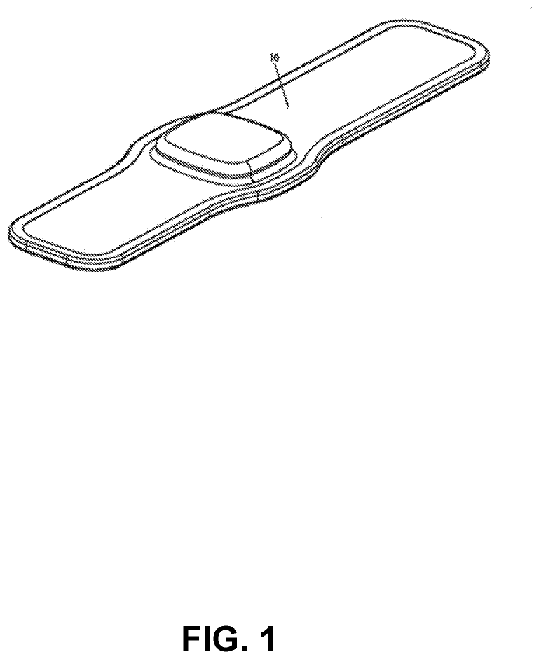

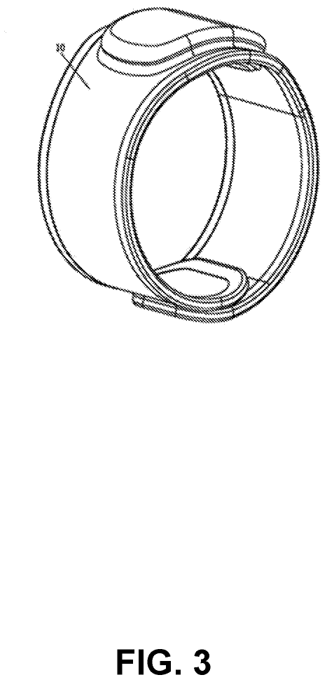

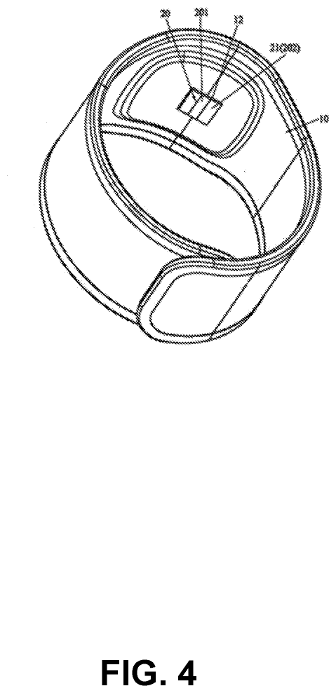

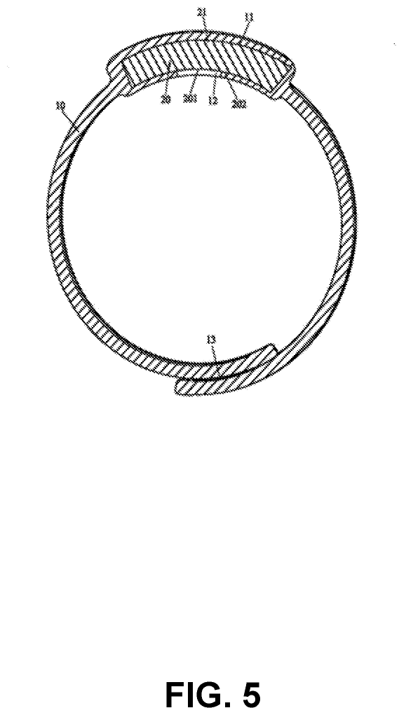

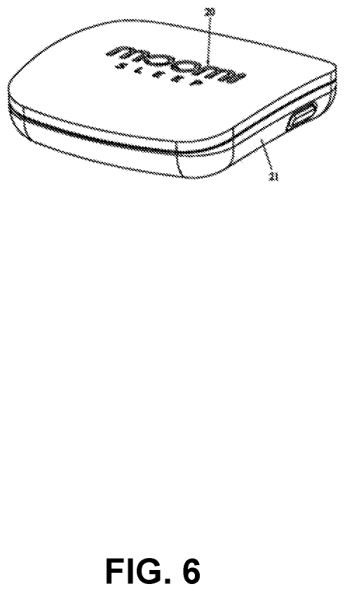

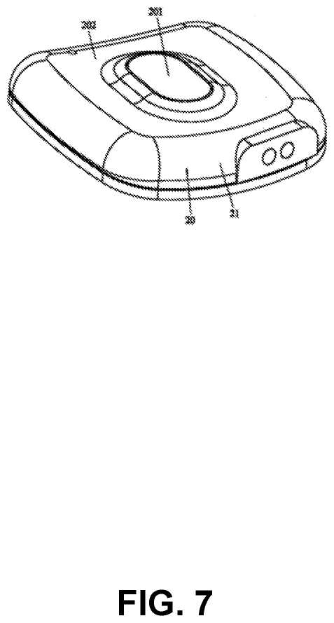

Refer to FIGS. 1 to 7, which show a specific structure of a preferred embodiment of the present invention, including a band 10 and a monitor host 20.

The side of the band 10 that is in contact with the human body is a textile. The band 10 has a receiving cavity 11 therein, and the side of the band 10 that is in contact with the human body is provided with a window 12 that communicates with the receiving cavity 11. In the present embodiment, the band 10 as a whole is an elastic textile and is made of pure cotton; the middle width of the band 10 is greater than the width of both ends thereof, and the two ends of the band 10 are fixedly connected by Velcro 13. The receiving cavity 11 is located at a middle position of the band 10.

The monitor host 20 is housed in the receiving cavity 11. The detection end 201 of the monitor host 20 is exposed through the window 12 and separated from the human body by textiles. The specific structure and working principle inside the monitor host 20 are all prior art, and the internal structure and working principle of the monitor host 20 are not described in detail here. Moreover, in the present embodiment, the monitor host 20 has a housing 21, and the bottom surface of the housing 21 is an arc-shaped concave surface 202 adapted to the human body, and the detection end 201 of the monitor host 20 is located on the arc-shaped concave surface 202.

The usage method of the present embodiment is detailed as follows:

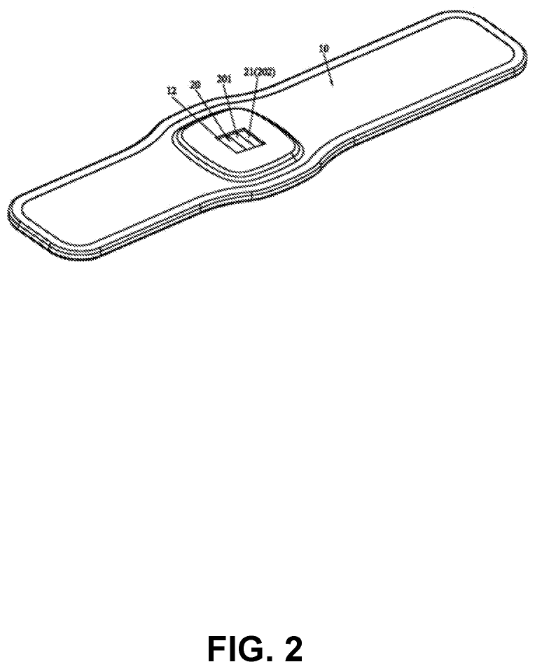

The present product is mainly used by children to monitor their body temperature, heart rate, and other human health indexes. During use, the monitor host 20 is inserted into the receiving cavity 11 through window 12 and is fixed thereto. As shown in FIG. 2, the detection end 201 of the monitor host 20 is exposed through the window 12, and the band is worn on the hand in such a way that the detection end 201 of the monitor host 20 faces the skin of the human body. Since the textile of the band 10 has a certain thickness, the monitor host 20 does not directly come in contact with the human body, providing good breathability to avoid skin allergies and the like. At this time, the detection end of the monitor host 20 can detect various human health indexes such as human body temperature, heart rate, and the like. These human body monitoring indexes are then transmitted to a mobile phone application via Bluetooth, and the received data is analyzed and sorted in the background to monitor changes in human health.

The design focus of the present utility model is: by designing the side of the band that is in contact with the human body as a textile and by coordinating with the monitor host in the receiving cavity, the monitor host can be separated from the human body by using the textile, effectively improving the breathability of the product and avoiding sweating, allergies, etc. that human skin is prone to, thus making the wearable more comfortable and very suitable for children.

The above is only a preferred embodiment of the present invention and does not limit the technical scope of the present invention in any way. Therefore, any minor modifications, equivalent changes, or modifications made to the above embodiment according to the technical essence of the present utility model will still belong to the scope of the technical solution of the utility model.

What is claimed is:

1. A wearable human health index monitor, comprising:
a band and a monitor host;
wherein a side of the band in contact with the human body is a textile, the band having a receiving cavity therein, wherein the side that is in contact with the human body is provided with a window that communicates with the receiving cavity; and
wherein the monitor host is housed in the receiving cavity, and the detection end of the monitor host is exposed through the window and separated from the human body by textiles.

2. The wearable human health index monitor according to claim 1, wherein the band is an elastic textile.

3. The wearable human health index monitor according to claim 1, wherein two ends of the band are fixedly connected by Velcro.

4. The wearable human health index monitor according to claim 1, wherein the receiving cavity is located at a middle position of the band.

5. The wearable human health index monitor according to claim 1, wherein the monitor host has a housing, and the bottom surface of the housing is an arc-shaped concave surface adapted to the human body.
